(19) United States
(12) Patent Application Publication
(10) Pub. No.: US 2026/0096882 A1
(43) Pub. Date: Apr. 9, 2026

(54) LARGE BORE SHEATH DEVICE INCLUDING COUPLING DEVICE

(71) Applicant: Silk Road Medical, Inc., Sunnyvale, CA (US)

(72) Inventors: Zach Tegels, Sunnyvale, CA (US); Kristen Lipschultz, Sunnyvale, CA (US); Adam Wigginton, Sunnyvale, CA (US); Brad Steele, Sunnyvale, CA (US); Stewart Kume, Sunnyvale, CA (US)

(73) Assignee: Silk Road Medical, Inc., Sunnyvale, CA (US)

(21) Appl. No.: 19/115,308

(22) PCT Filed: Sep. 28, 2023
(86) PCT No.: PCT/US2023/033949

§ 371 Date: Mar. 26, 2025

Related U.S. Application Data
(60) Provisional application No. 63/377,833, filed on Sep. 30, 2022.

(51) Int. Cl. A61F 2/24

(52) U.S. Cl. CPC A61F 2/2436

(57) ABSTRACT

Various embodiments of a sheath device and related methods are described herein. In some embodiments, the sheath device can include a sheath hub and a sheath body extending from the sheath hub. The sheath body can include an inner passageway and a proximal section having a first length and a first flexibility along the first length. The sheath body can further include a distal section having a second length and a second flexibility along the second length, and the second flexibility can be greater than the first flexibility. The sheath body can further include an atraumatic tip positioned at a distal end of the sheath body, and the atraumatic tip can include a rounded distal end.

### CROSS REFERENCE TO RELATED APPLICATION

This application claims priority to U.S. Application Ser. No. 63/377,833, filed Sep. 30, 2022, titled “LARGE BORE SHEATH DEVICE INCLUDING COUPLING DEVICE,” the contents of which are fully incorporated by reference.

### BACKGROUND

Various medical procedures involve accessing inner lumens of vessels. Sheaths or catheters can provide a conduit from an extracorporeal space to the inside of the body, such as within vessels of the body. Devices may be used to insert tools and/or implants into the body and/or provide therapy during various procedures, such as through a variety of access sites (e.g. surgical, percutaneous, or existing body orifices).

During large bore access of vessels, such as the femoral artery, axillary artery, and common carotid artery, sheath sizes ranging from approximately 10 French (F) to approximately 21 F may be used. A surgical cut-down or cut-down with anastomosis connection and artery wall resection may be employed for direct vessel access, such as instead of blunt tissue dissection. The distance between a deployment site and access site can be shorter than femoral access and may involve less tortuosity. Some current sheaths used for large bore access were developed for percutaneous femoral access, apical access, or direct aortic access. At least some of such sheaths do not allow for optimal access with other vessels, including the femoral artery. For example, at least some currently available sheaths can include unfavorable flexibility profiles, tip shapes, tip softness, and/or hub designs.

Some current sheaths can have functional and/or structural limitations when there is a need for blunt tissue dissection during percutaneous access. While the immediate site of access in the femoral artery, axillary artery, and carotid artery can be free of disease, some vascular locations (e.g., ostial lesion, aortic arch, etc.) along where the sheath may need to traverse can have areas of calcification and disease that can cause an embolic event or arterial dissection.

Additionally, devices such as prosthetic heart valves, transcatheter heart valve repair devices, intra atrial shunting devices, and other devices can require both radial and axial alignment for proper deployment and permanent implantation. The delivery system is typically used to align devices with a sheath that serves as a conduit for delivery of devices and the sheath may not assist with axial or radial alignment of devices, such as relative to surrounding vasculature.

### SUMMARY

Aspects of the current subject matter include various embodiments of a sheath device and related methods. In an aspect, various embodiments of a sheath device are described that include a sheath hub and a sheath body extending from the sheath hub. The sheath body can include an inner passageway and a proximal section having a first length and a first flexibility along the first length. The sheath body can further include a distal section having a second length and a second flexibility along the second length, and the second flexibility can be greater than the first flexibility. The sheath body can further include an atraumatic tip positioned at a distal end of the sheath body, and the atraumatic tip can include a rounded distal end.

In some variations one or more of the following features can optionally be included in any feasible combination. The sheath device can further include a transition section positioned between the proximal section and the distal section, and the transition section can have a third length and a third flexibility that is less than the first flexibility and greater than the second flexibility. The sheath body can include more than one distal section and more than one proximal section. The distal sections and the proximal sections can alternate along a length of the sheath body. The sheath device can further include a support structure that extends along a length of the sheath body, and the atraumatic tip can be void of the support structure. The support structure can include a coil having variable pitch coiling and/or a variable number of wires including the coil along the length of the sheath body. The support structure can include a braid having variable programmable picks per inch along the length of the sheath body.

The sheath body can include a radiopaque element positioned along the distal end of the sheath body and proximal to the atraumatic tip. The sheath hub can include one or more of a flush port and a hemostasis valve. The sheath hub can be at least party formed of a translucent material adjacent the flush port for allowing viewing of internal components and/or fluid within the sheath hub. The sheath hub can include a conduit adapter that is configured to couple to the sheath body and allow a conduit to couple to the sheath hub. The conduit adapter can include a tubular body with at least one groove along an outer surface of the tubular body. Each groove of the at least one groove can include a groove length that allows a coupling element to secure the conduit to the tubular body within the groove length. The sheath body can be approximately 10 French (F) to approximately 36 F.

The sheath hub can include a flexible extension including a coupling device. The coupling device can include a coupling body, a cap releasably couplable to the coupling body, and a collet having a collet passageway that reduces in diameter when the collet is secured between the coupling body and the cap to thereby secure a part of a delivery device extending along the collet passageway. The coupling device can axially and radially secure the delivery device to the sheath device when the coupling device is in a secured configuration.

In another interrelated aspect of the current subject matter, a method of a sheath device is described that includes advancing a part of the sheath device in an inner lumen of a vessel. For example, the sheath device can include a sheath hub and a sheath body extending from the sheath hub. The sheath body can include an inner passageway and a proximal section having a first length and a first flexibility along the first length. The sheath body an further include a distal section having a second length and a second flexibility along the second length, and the second flexibility can be greater than the first flexibility. The sheath body can further include an atraumatic tip positioned at a distal end of the sheath body, and the atraumatic tip can include a rounded distal end. The method can further include positioning the distal section of the sheath body along the inner lumen of the vessel.

In some variations one or more of the following features can optionally be included in any feasible combination. The sheath device can further include a transition section positioned between the proximal section and the distal section, and the transition section can have a third length and a third flexibility that is less than the first flexibility and greater than the second flexibility. The sheath body can include more than one distal section and more than one proximal section. The distal sections and the proximal sections can alternate along a length of the sheath body. The sheath device can further include a support structure that extends along a length of the sheath body, and the atraumatic tip can be void of the support structure. The support structure can include a coil having variable pitch coiling and/or a variable number of wires including the coil along the length of the sheath body. The support structure can include a braid having variable programmable picks per inch along the length of the sheath body.

The sheath body can include a radiopaque element positioned along the distal end of the sheath body and proximal to the atraumatic tip. The sheath hub can include one or more of a flush port and a hemostasis valve. The sheath hub can be at least party formed of a translucent material adjacent the flush port for allowing viewing of internal components and/or fluid within the sheath hub. The sheath hub can include a conduit adapter that is configured to couple to the sheath body and allow a conduit to couple to the sheath hub. The conduit adapter can include a tubular body with at least one groove along an outer surface of the tubular body. Each groove of the at least one groove can include a groove length that allows a coupling element to secure the conduit to the tubular body within the groove length. The sheath body can be approximately 10 French (F) to approximately 36 F.

The sheath hub can include a flexible extension including a coupling device. The coupling device can include a coupling body, a cap releasably couplable to the coupling body, and a collet having a collet passageway that reduces in diameter when the collet is secured between the coupling body and the cap to thereby secure a part of a delivery device extending along the collet passageway. The coupling device can axially and radially secure the delivery device to the sheath device when the coupling device is in a secured configuration.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 illustrates a partially exploded side view of an embodiment of an access device including a sheath for providing access to an inner lumen of a vessel.

FIG. 2 illustrates a side partial view of the access device of FIG. 1 including a sheath stopper.

FIG. 3 illustrates an example of the access system of FIG. 1 accessing an inner lumen of a vessel.

FIG. 4 illustrates a top view of an embodiment of a sheath device.

FIG. 5 illustrates a partial cross-section view of a distal end of a sheath body of an embodiment of the sheath device of FIG. 4.

FIG. 6 illustrates a side view of an embodiment of the sheath body of FIG. 4.

FIG. 7A illustrates a side view of an embodiment of the sheath device of FIG. 4 including a conduit connector having a plurality of grooves.

FIG. 7B illustrates a partial cross-section view of an embodiment of the sheath device of FIG. 4 showing a anastomosis clamp coupling a conduit to the conduit connector.

FIG. 7C illustrates a top perspective view of an embodiment of the sheath device of FIG. 4 showing the conduit coupled to the conduit connector and a vessel.

FIG. 8 illustrates a partially transparent exploded side view of an embodiment of a coupling device.

When practical, similar reference numbers denote similar structures, features, or elements.

### DETAILED DESCRIPTION

The disclosed methods, apparatus, and systems are directed to various embodiments of a sheath device for large bore procedures. Additionally, the sheath devices described herein can be used with a variety of delivery systems and access systems. Embodiments of the sheath device can be configured to assist with large bore sheath access into various vessels, such as the femoral artery, axillary artery, and common carotid artery.

For example, large bore sheath access can include sheath sizes that are approximately 10 French (F) to approximately 21 F. Large bore sheath access using at least some currently available sheaths can result in vascular trauma and injury to the patient, such as due to puncturing of the vessel and/or creating embolic material. The sheath devices described herein can safely and effectively provide large bore sheath access into various vessels.

Additionally, at least some currently available large bore sheath devices are not optimized for deairing. The sheath devices described herein can include a single sheath hub that efficiently and effectively performs deairing. Furthermore, axial and radial alignment of delivery devices relative to sheath devices and/or a vessel can be important for some procedures. Embodiments of a coupling device for efficiently axially and radially securing a delivery device to a sheath device are disclosed herein.

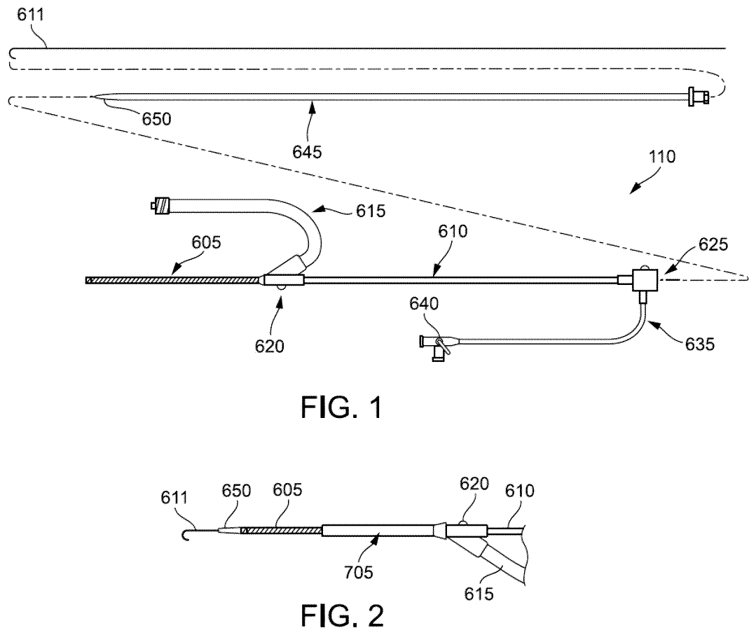

FIG. 1 shows an exemplary embodiment of an access system 110 including a sheath 605, a proximal extension 610, a flow line 615, an adaptor or Y-connector 620, and a hemostasis valve 625. The access system 110 may also comprise a dilator 645 with a tapered tip 650 and an introducer guide wire 611. The access system 110 together with the dilator 645 and introducer guidewire 611 can be used together to gain access to a vessel. Features of the access system may be optimized for access into a variety of vessels, such as via transfemoral and transcarotid access.

The sheath 605 can be adapted to be introduced through an incision or puncture in a wall of a vessel (e.g., femoral artery, common carotid artery, etc.), either an open surgical incision or a percutaneous puncture established, for example, using the Seldinger technique. As shown in FIG. 1, the proximal extension 610, which includes an elongated body, can have an inner lumen that is contiguous with an inner lumen of the sheath 605. The lumens can be joined by the Y-connector 620 that also connects a lumen of the flow line 615 to the sheath. For example, the flow line 615 can connect to and form a first leg of a retrograde shunt.

A flush line 635 can be connected to the side of the hemostasis valve 625 and can have a stopcock 640 at its proximal or remote end. The flush-line 635 can allow for the introduction of saline, contrast fluid, or the like, during the procedures. The flush line 635 can also allow pressure monitoring during the procedure. A dilator 645 having a tapered distal end 650 can be provided, for example, to facilitate introduction of the sheath 605 into the vessel. The dilator 645 can be introduced through the hemostasis valve 625 so that the tapered distal end 650 extends through the distal end of the sheath 605, as best seen in FIG. 2. The dilator 645 can have a central lumen to accommodate a guide wire. Typically, the guide wire is placed first into the vessel, and the dilator/sheath combination travels over the guide wire as it is being introduced into the vessel. Optionally, a sheath stopper 705 such as in the form of a tube may be provided which is coaxially received over the exterior of the sheath 605, also as seen in FIG. 2.

The access system 110 can provide access to an inner lumen of a vessel, such as for performing at least part of any number of a variety of procedures. For example, during a transcarotid artery revascularization (TCAR) procedure, the sheath 605 can be inserted into the common carotid artery (CCA) of the patient. To achieve reverse flow of blood, the CCA may be occluded to stop antegrade blood flow from the aorta through the CCA. Flow through the CCA can be occluded with an external vessel loop or tape, a vascular clamp, an internal occlusion member such as a balloon, or other type of occlusion means. One or more of a variety of blood flow control can be used during a procedure using the access system 110.

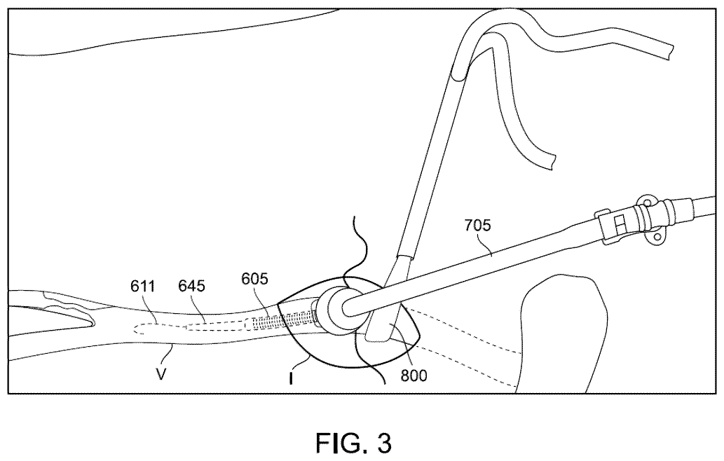

FIG. 3 illustrates an embodiment of an access system 110 including an embodiment of the sheath 605 inserted within a vessel V, such as the carotid artery, exposed through an incision I. For example, the sheath stopper 705 can be used with the sheath 605 to assist with guiding and positioning the sheath 605 within the vessel V, as shown in FIG. 3. The sheath guidewire 611 and dilator 645 can protrude out from a distal opening of the sheath 605. For example, manual occlusion of the vessel V by a clinician at an occlusion location proximal to the distal tip of the sheath 605 may be provided from the outside of the vessel V using a vascular clamp 800, such as a Rummel tourniquet or vessel loop positioned proximal to the sheath insertion site. Occlusion of the vessel can form a closed section of the vessel along which blood is prevented from flowing. Once the target vessel V has been treated by the operator, the vascular clamp 800 can be released permitting resumption of antegrade blood flow. One or more of a variety of ways can be used to occlude the vessel during a procedure without departing from the scope of this disclosure.

Various embodiments of a sheath device that can be used with a variety of delivery systems and access systems, including the access system 110 and procedures described herein, are described in detail below.

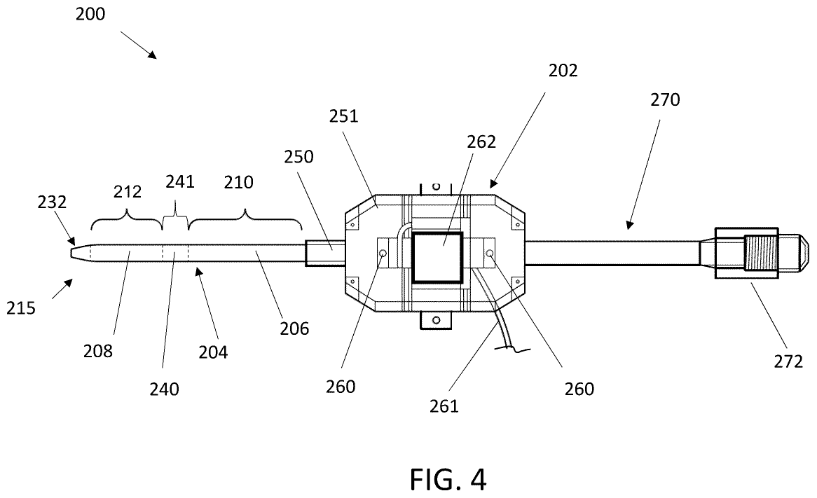

FIG. 4 illustrates an embodiment of a sheath device 200 including a sheath hub 202 and a sheath body 204 extending from the sheath hub 202. The sheath body 204 can include a tubular body having at least two sections extending circumferentially and lengthwise along a part of the sheath body 204, and the at least two sections can include at least two different flexibilities. As such, the sheath body 204 can have more than one flexibility along its length. For example, as show in FIG. 4, the sheath body 204 can include a proximal section 206 that extends from and/or is coupled to the sheath hub 202. The sheath body 204 can also include a distal section 208 that extends along the sheath body 204 at a location that is further away from the sheath hub 202 compared to the proximal section 206.

For example, the proximal section 206 can extend a proximal length 210 and have a first flexibility, and the distal section 208 can extend a distal length 212 and have a second flexibility that is greater than the first flexibility of the proximal section 206. As such, the distal section 208 can be more flexible compared to the proximal section 206. This can allow the proximal section 206 to provide support and greater stiffness (e.g., compared to the distal section 208) for safely and effectively performing a variety of procedures, such as delivery of a heart valve along a vessel (e.g., transcatheter aortic valve replacement (TAVR)). For example, the combination of a more flexible distal section 208 and a less flexible proximal section 206 of the sheath body 204 can gently and atraumatically assist with straightening vessels with minimal risk of vessel damage (e.g., vessel dissection), creating and/or breaking away embolic material in the vessel.

In some embodiments, the distal length 212 of the distal section 208 can have a length that is approximately 2 centimeters (cm) to approximately 15 cm. In some embodiments, the proximal length 210 of the proximal section 206 can have a length that is approximately 8 centimeters cm to approximately 40 cm. In some embodiments, the distal section 208 can be configured to have a measured Shore Hardness of approximately 40A to approximately 80A. In some embodiments, the proximal section 206 can be configured to have a measured Shore Hardness of approximately 40D to approximately 82D. Other section lengths and material properties (e.g., Shore Hardness, flexibility, etc.) of the sheath body are within the scope of this disclosure. In some embodiments, the sheath body is approximately 10 French (F) to approximately 36 F.

In some embodiments, the proximal section 206 and distal section 208 can be formed out of the same or different materials. For example, the proximal section 206 can be formed out of a material that has lower flexibility compared to material forming the distal section 208. In some embodiments, the proximal section 206 and distal section 208 can be formed out of the same or similar material and the distal section 208 can have a thinner sheath tubular body wall compared to the proximal section 206, thus allowing the distal section 208 to be more flexible than the proximal section 206. For example, wall thicknesses of the tubular sheath body 204 can vary along the proximal section 206 and/or distal section 208 for achieving desired flexibility variance, such as wall thicknesses that are approximately 0.008 inch to approximately 0.025 inch.

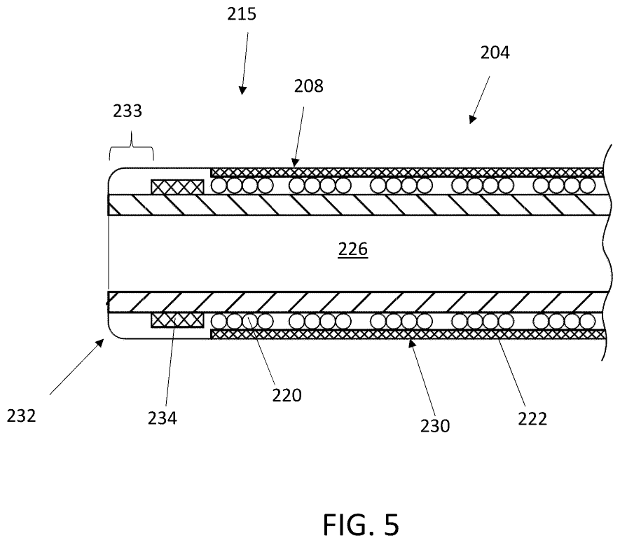

FIG. 5 illustrates a cross-section view of a part of the sheath body 204 including a distal end 215 of the sheath body 204. As shown in FIG. 5, in some embodiments at least a part of the sheath body 204 can include at least one support structure, such as a coil 220 and/or a braid 222. For example, an embodiment of the coil 220 can extend along at least a part of the sheath body 204, such as along a part of the distal section 208, as shown in FIG. 5. In some embodiments, the coil 220 can extend along a length of the proximal section 206 and/or the distal section 208, such as along the entire length or a part of the proximal section 206 and/or distal section 208. In some embodiments, one or more coils 220 can extend circumferentially along the sheath body 204, such as coiled around a tubing liner 224 that forms an inner passageway 226 of the sheath body 204. For example, the tubing liner 224 can be formed of a polytetrafluoroethylene (PTFE) material and can extend along all or part of the length of the sheath body 204.

In some embodiments, the coil 220 can be wound with more than one wire (e.g., multifilar coil) and the distal section 208 can include a length of the coil 220 including less filars or wires along the distal section 208 compared to the proximal section 206, thereby allowing the distal section 208 to be more flexible compared to the proximal section 206. For example, the coil 220 can include 4 filars or wires along the distal section and 5 or more filars or wires along the proximal section. In some embodiments, the sheath body 204 can include variable pitch coiling of the coil 220, such as the coil 220 having a greater pitch coiling along the distal section 208 compared to the proximal section 206 thereby allowing the distal section 208 to be more flexible compared to the proximal section 206. In some embodiments, the pitch coiling can be approximately 0.020 inch (in) to approximately 0.016 in. For example, the coil 220 can have an approximately 0.0020 in to an approximately .0040 in diameter, such as an approximately 0.0030 in diameter wire (e.g., roundwire coil).

As shown in FIG. 5, some embodiments of the sheath body 204 can include one or more of a variety of braids 222 extending along at least one of the proximal section 206 and/or the distal section 208. In some embodiments, the braid 222 can extend circumferentially around the coil 222, such as between an outer jacket 230 and the coil 222 and/or inner liner 224. For example, the braid 222 can include variable programmable picks per inch (PPI) and/or density along a length. In some embodiments, the braid 222 can have a greater PPI along the proximal section 206 compared to the distal section 208 thereby allowing the distal section 208 to be more flexible compared to the proximal section 206. One or more of a variety of materials can be used to form the coil 220 and/or the braid 224, such as stainless steel, polyetheretherketone (PEEK), fluorinated ethylene propylene (FEP), and polyvinylidene fluoride (PVDF). In some embodiments, the sheath body 204 can be made out of one or more of a polymer material. For example, the outer jacket 230 can be made out of one or more polymer materials. Other materials for forming any part of the sheath body 204 are within the scope of this disclosure.

In some embodiments, the sheath body 204 can include at least one tie layer (e.g., thin thermoplastic coating), such as along at least one of the proximal section 206 and the distal section 208. For example, the tie layer can be placed along the proximal section to assist with allowing the distal section 208 to be more flexible compared to the proximal section 206.

As shown in FIGS. 4 and 5, the sheath body 204 can include an atraumatic tip 232. For example, the atraumatic tip 232 can be positioned distal to the distal section 208. For example, the atraumatic tip 232 can have a tapered or radiused distal end to provide an atraumatic effect against tissue (e.g., vessels) and improve ease of insertion of the distal end of the sheath body, such as compared to a distal end of the sheath body having a blunt end with sharp edges. In some embodiments, the atraumatic tip 232 can include a tip length 233 that is approximately 1 millimeter (mm) to approximately 5 mm. As shown in FIG. 5, the atraumatic tip 232 can be void of the coil 220 and/or braid 222.

As shown in FIG. 5, the sheath body 204 can include a radiopaque element 234 that is viewable under x-ray. For example, the radiopaque element 234 can include a structural reinforcement element that includes a radiopaque marker and extends along a length of the distal end 215, such as along a distal part of the distal section 208. In some embodiments, the radiopaque element 234 is positioned adjacent the atraumatic tip 232 such that the atraumatic tip 232 is void of the radiopaque element 234. In some elements, the atraumatic tip 232 can include a radiopaque element 234, such as an embodiment of the radiopaque element that does not provide structural reinforcement.

As shown in FIG. 4, some embodiments of the sheath body 204 can include at least one transition section 240 that extends a transition length 241 and is positioned between the proximal section 206 and the distal section 208. For example, the transition section 240 can be formed out of the same or different material as the proximal section 206 and/or the distal section 208. The transition length 241 can be approximately 3 cm to approximately 7 cm, such as approximately 5 cm. For example, the transition section 240 can be formed of a material having a Shore Hardness of approximately 90A to approximately 35D.

In some embodiments, the sheath body can include more than one proximal section 206 and/or more than one distal section 208. For example, such additional proximal section 206 and/or distal section 208 can provide a more complex flexibility profile along the length of the sheath body 204, such as for navigating around more complex anatomy.

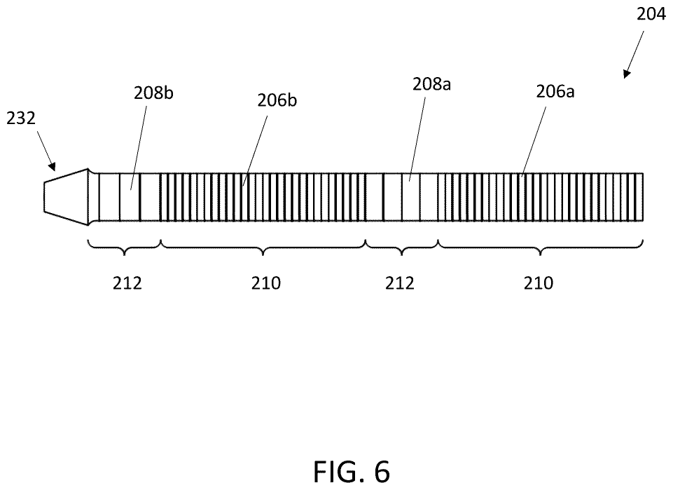

FIG. 6 illustrates an embodiment of a sheath body 204 including more than one proximal section 206 extending a proximal length 210 and more than one distal section 208 extending a distal length 212. The proximal lengths 210 can be the same or different, as well as the distal lengths 212 can be the same or different. As shown in FIG. 6, the sheath body 204 can include a first proximal section 206a extending from and/or coupled to the sheath hub 202, a first distal section 208a extending distal to the first proximal section 206a, a second proximal section 206b extending distal to the first distal section 208a, and a second distal section 208b extending distal to the second proximal section 206b. An embodiment of the atraumatic tip 232 can be positioned distal to the second distal section 208b, as shown in FIG. 6. In some embodiments, one or more transition sections, such as transition section 240 in FIG. 4, can extend between any one or more of the distal sections 208 and proximal sections 206.

As shown in FIG. 4, the sheath hub 202 of the sheath device 200 can include a conduit connector 250 that extends from a housing 251 of the sheath hub 202. For example, the conduit connector 250 can assist with coupling the sheath body 204 to the sheath hub 202 and place the inner passageway 226 of the sheath body 204 in fluid communication with one or more fluid lines. For example, the sheath hub 202 can include and/or be coupled to at least one hemostasis valve 260, a flush port 260, and a flexible extension 270. The sheath hub 202, flush port 260, and flexible extension 270 can provide a fluid pathway to and/or from the inner passageway 226 of the sheath body 204. In some embodiments, the sheath hub 202 can include two or more hemostasis valves 260.

In some embodiments, the sheath hub 202 can include a viewing window 262 (e.g., part of housing 251 formed of translucent material) that can allow a user to view at least one fluid pathway extending along the sheath hub 202, such as for efficiently and effectively deairing the sheath device 200 (e.g., by confirming removal of air from sheath device 200). In some embodiments, the flexible extension 270 can include a coupling device 272 that allows the flexible extension 270 (e.g., tubing) to couple to a delivery device and/or fluid line, such as for delivering and/or removing fluids to and/or from the sheath device, respectively. For example, the flexible extension 270 can assist with flushing the sheath device 200 with a saline or other media and/or saturating with carbon dioxide.

In some embodiments, the conduit connector 250 is configured to allow a conduit to be coupled thereto, such as for coupling the conduit to the sheath device 200 prior to advancing the sheath body 204 into a vessel. In some embodiments, the conduit connector 250 can be approximately 1 cm to approximately 12 cm in length. In some embodiments, the conduit connector 250 can include an adjustable length feature. For example, the adjustable length of the conduit connector 250 can allow and/or facilitate sheath placements either at a deeper or more shallow position into the artery or vessel. The adjustability of the conduit connector 250 can also reduce the overall length of the of the conduit connector 250.

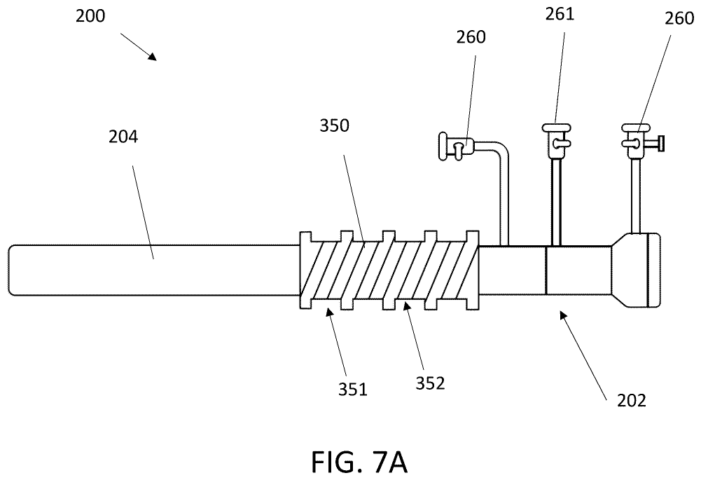

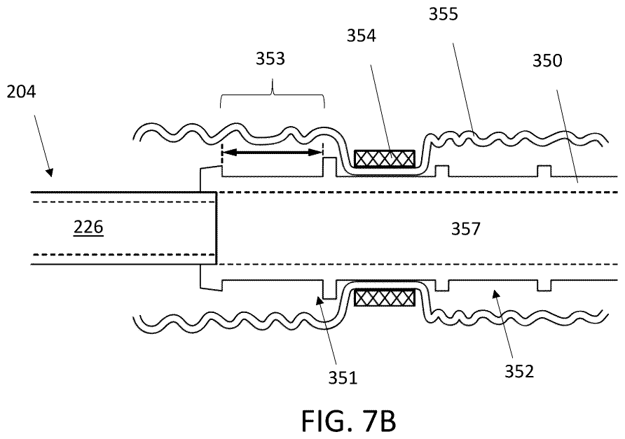

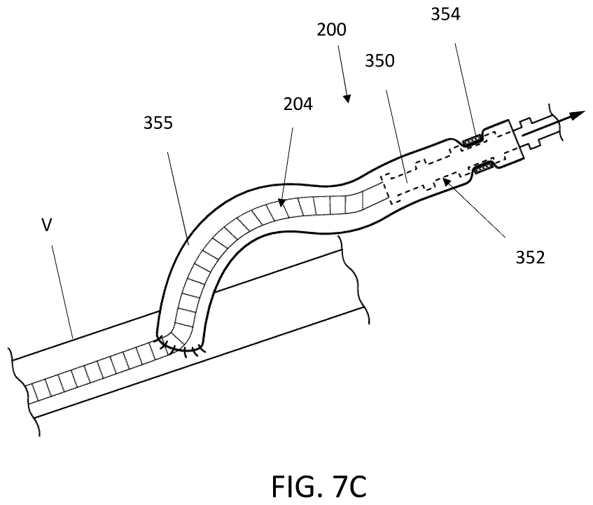

FIGS. 7A-7C illustrate an embodiment of the sheath device 200 including an embodiment of the conduit connector 350 that includes a plurality of grooves 352 for allowing a conduit 355 to be securely coupled to the conduit connector 350. As shown in FIGS. 7A and 7B, the conduit connector 350 can have a tubular body 351 with a coupling passageway 357 extending through the tubular body 351. As shown in FIG. 7A, the conduit connector 350 can extend from the sheath hub 202 and couple to the sheath body 204, such as receive a part of the sheath body 204 in the coupling passageway 357, as shown in FIG. 7B. The coupling passageway 357 can be in fluid communication with one or more of the inner passageway 226 of the sheath body 204, the flexible extension, 270, the at least one hemostasis valve 260, and the flush port 261. In some embodiments, the conduit connector 350 can provide strain relief. For example, the conduit connector 350 can function in a manner similar to a catheter strain relief where the conduit connector 350 relieves or provides a transition between the rigid sheath hub 202 or handle and the more flexible sheath body 204. The material and material properties of the conduit connector 350 can depend on the interface between the sheath hub 202 and sheath body 204. As such, embodiments of the conduit connector 350 providing strain relief can be fabricated from one or more of a variety of material in a variety of geometries.

As shown in FIG. 7B, the conduit connector 350 can include more than one groove 352 (e.g., four or more grooves 352) formed along an outer wall of the tubular body 351. Each groove 352 can extend a groove length 353 along the length of the tubular body 351. The groove length 353 can be sized to allow a coupling element 354 (e.g., anastomosis clamp) to secure to the outer wall of the tubular body 351 and within a groove 352 (e.g., within a groove length 353). Additionally, the coupling element 354 can secure a part of the conduit 355 to the conduit connector 350, such as within a groove 352, as shown in FIGS. 7B and 7C. For example, the groove length 353 can be approximately 0.75 cm to approximately 1.25 cm, such as 1 cm. The depth of the groove 352 can be approximately 0.075 cm to approximately 0.125 cm, such as 0.1 cm. For example, the conduit 355 can include an anastomosis formed of one or more of a polyethylene terephthalate (PET) and PTFE material.

As shown in FIG. 7C, the conduit 355 can be secured at a first end to the conduit connector 350 using the coupling element 354 (e.g., hemostatic clamp, anastomosis clamp). Additionally, a second end of the conduit 355 can be secured to a vessel V (e.g., common carotid artery, femoral artery, etc.). For example, the second end of the conduit 355 can be sutured to the vessel V via a cut-down access to the vessel V. As shown in FIG. 7C, the sheath body 204 can be coupled to the first end of the conduit 355 and allowed to extend into the vessel V, including at variable insertion lengths. Various procedures can be safely and efficiently performed using the sheath device 200 coupled to an embodiment of the conduit 355 that is secured to a vessel V, such as during an endovascular procedure using a conduit (e.g., TCAR, thoracic endovascular aortic repair (TEVAR), TAVR, etc.).

The sheath device 200 can be coupled to a variety of conduits, devices, and/or fluid pathways. For example, as shown in FIG. 4, the sheath hub 202 can include a flexible extension with a coupling device 272 configured to couple to a delivery device and/or fluid line. In some embodiments, the coupling device 272 can be configured to radially and axially align a device (e.g., delivery device, etc.) to the sheath device 200.

For example, axial alignment can be important for TAVR devices in order to prevent permanent pacemaker implant when the valve is delivered too deep. Radial alignment can be important for TAVR devices to provide optimal commissural alignment to preserve coronary access and permit implantation of a second valve in the future. Furthermore, Transcatheter Tricuspid and Mitral Valve replacement implants may have a non-symmetrical cross-sectional shape that requires specific radial orientation for optimal implantation. Over long distances, the delivery system may not be able to transfer sufficient torque to push or provide optimal radial or axial alignment. As such, various embodiments of the coupling device 272 are disclosed that provides variable and controlled torque and axial coupling of a delivery device to a delivery sheath (e.g., sheath device 200) to achieve improved control of radial and axial alignment of at least the delivery devices and delivery sheath (e.g., sheath device 200).

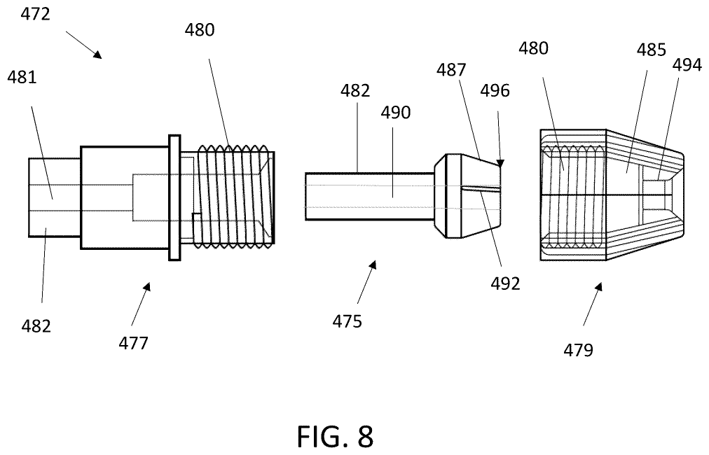

FIG. 8 illustrates an embodiment of the coupling device 472 including a collet 475 that is captured and secured between a coupling body 477 and a cap 479. For example, the coupling body 477 can be securely coupled to a part of a delivery sheath, such as the flexible extension 270 of the sheath device 200. For example, the flexible extension 270 can be inserted into and coupled to a body passageway 481 that extends through the coupling body 477 and/or can couple to an outer wall 482 of the coupling body 477. The coupling body 477 and cap 479 can include threads 480 that threadably engage and secure the cap 479 to the coupling body 477. Additionally the coupling body 477 can allow an extension 482 of the collet 475 to slidably engage and be inserted into a part of the body passageway 481.

In some embodiments, the cap 479 can include a recess 485 that is shaped to conform to at least a part of a head 487 of the collet 475. As such, when the extension 482 of the collet 475 is inserted within the body passageway 581 and the head 487 of the collet 475 is aligned with and/or positioned within the cap 479 the cap 479 can threadably engage the coupling body 477 (e.g., via threads 480) to secure the cap 479 to the coupling body 477 and the collet 475 between and within the cap 479 and coupling body 477, which is referred to herein as the secured configuration of the coupling device 472. Once the coupling device 472 is in the secured configuration, the collet 475 is axially and rotationally locked in position relative to the cap 479 and coupling body 477.

As shown in FIG. 8, the collet 475 can include a collet passageway 490 that extends along the length of the collet 475 and can be approximately aligned with the body passageway 481 and a cap passageway 494 of the cap 479, such as when the coupling device 472 is in the secured configuration. The head 487 of the collet 475 can include a space 492 along the wall forming the head 487, such as shown in FIG. 8. The space 492 can allow an inner diameter of the collet passageway 490 along a receiving end 496 of the collet 475 to change dimensions. For example, formation of the secured configuration can cause the head 487 of the collet 475 to advance into the recess 485 of the cap 475, and the recess 485 can be tapered inward such that advancement of the head 487 into the recess 485 causes at least the receiving end 496 of the collet 475 to reduce in diameter, such as by narrowing and/or closing the space 492. This also causes the collet passageway 490 to reduce in diameter.

In some embodiments, a tubing or part of a delivery device can extend through the cap passageway 494 and at least partly through the collet passageway 490 to allow the head 487 of the collet 475 to secure around the part of the delivery device when the coupling device forms the secured configuration (e.g., due to radial force and friction force by collet passageway 490 along the part of the delivery device). Prior to formation of the secured configuration, a desired axial and radial alignment of the coupling device 472 and sheath device (e.g., sheath device 200) can be formed relative to the delivery device. Once in the desired axial and radial alignment, the cap 479 can be coupled to the coupling body 477 without disrupting the axial and/or radial alignment. As such, the delivery device can be efficiently and effectively secured in the desired axial and radial alignment relative to the sheath device 200 for performing a variety of procedures, such as a TAVR procedure.

Other coupling device embodiments are within the scope of this disclosure. For example, the collet can be replaced with a collapsible braid or weave that can transition from a larger inner diameter to a smaller inner diameter by rotating an embodiment of the cap 479 relative to an embodiment of the coupling body 477. For example, the braid or weave can be formed of a stainless steel, polymer and/or a combination of materials. The radial force and friction force of the braid along a part of the delivery system can, for example, allow secure coupling between a sheath (e.g., the sheath device 200) and a delivery system.

While these descriptions contain many specifics, these should not be construed as limitations on the scope of an invention that is claimed or of what may be claimed, but rather as depictions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore, the spirit and scope of the appended claims should not be limited to the description of the embodiments contained herein.

1. A sheath device, comprising:
a sheath hub;
a sheath body extending from the sheath hub, the sheath body comprising:
an inner passageway;
a proximal section having a first length and a first flexibility along the first length;
a distal section having a second length and a second flexibility along the second length, wherein the second flexibility is greater than the first flexibility; and
an atraumatic tip positioned at a distal end of the sheath body, the atraumatic tip including a rounded distal end.

2. The sheath device of claim 1, further comprising:
a transition section positioned between the proximal section and the distal section, the transition section having a third length and a third flexibility that is less than the first flexibility and greater than the second flexibility.

3. The sheath device of claim 2, wherein the sheath body includes more than one distal section and more than one proximal section.

4. The sheath device of claim 3, wherein the distal sections and the proximal sections alternate along a length of the sheath body.

5. The sheath device of claim 1, wherein a support structure extends along a length of the sheath body, the atraumatic tip being void of the support structure.

6. The sheath device of claim 5, wherein the support structure includes a coil having variable pitch coiling and/or a variable number of wires comprising the coil along the length of the sheath body.

7. The sheath device of claim 5, wherein the support structure includes a braid having variable programmable picks per inch along the length of the sheath body.

8. The sheath device of claim 1, wherein the sheath body includes a radiopaque element positioned along the distal end of the sheath body and proximal to the atraumatic tip.

9. (canceled)

10. The sheath device of claim 1, wherein the sheath hub is at least party formed of a translucent material for allowing viewing of internal components and/or fluid within the sheath hub.

11. The sheath device of claim 1, wherein the sheath hub includes a conduit adapter that is configured to couple to the sheath body and allow a conduit to couple to the sheath hub.

12. The sheath device of claim 11, wherein the conduit adapter includes a tubular body with at least one groove along an outer surface of the tubular body.

13. The sheath device of claim 12, wherein each groove of the at least one groove includes a groove length that allows a coupling element to secure the conduit to the tubular body within the groove length.

14. (canceled)

15. The sheath device of claim 1, wherein the sheath hub comprises a flexible extension including a coupling device.

16. The sheath device of claim 15, wherein the coupling device comprises:
a coupling body;
a cap releasably couplable to the coupling body; and
a collet having a collet passageway that reduces in diameter when the collet is secured between the coupling body and the cap to thereby secure a part of a delivery device extending along the collet passageway.

17. The sheath device of claim 16, wherein the coupling device axially and radially secures the delivery device to the sheath device when the coupling device is in a secured configuration.

18. A method of a sheath device, comprising:
advancing a part of the sheath device in an inner lumen of a vessel, the sheath device comprising:
a sheath hub;
a sheath body extending from the sheath hub, the sheath body comprising: an inner passageway;
a proximal section having a first length and a first flexibility along the first length;
a distal section having a second length and a second flexibility along the second length, wherein the second flexibility is greater than the first flexibility; and
an atraumatic tip positioned at a distal end of the sheath body, the atraumatic tip including a rounded distal end; and
positioning the distal section of the sheath body along the inner lumen of the vessel.

19. The method of claim 18, wherein the sheath body further comprises a transition section positioned between the proximal section and the distal section, the transition section having a third length and a third flexibility that is less than the first flexibility and greater than the second flexibility.

20-21. (canceled)

22. The method of claim 18, wherein a support structure extends along a length of the sheath body, the atraumatic tip being void of the support structure.

23. The method of claim 22, wherein the support structure includes a coil having variable pitch coiling and/or a variable number of wires comprising the coil along the length of the sheath body.

24. The method of claim 22, wherein the support structure includes a braid having variable programmable picks per inch along the length of the sheath body.

25-34. (canceled)
